(19) United States
(12) Patent Application Publication
(10) Pub. No.: US 2026/0068856 A1
(43) Pub. Date: Mar. 12, 2026

(54) NON-CONTACT VARIABLE SENSOR SYSTEM FOR ANIMALS

(71) Applicant: Zomedica Inc., Ann Arbor, MI (US)

(72) Inventors: Greg Blair, Ann Arbor, MI (US); Kenneth Balogh, Ann Arbor, MI (US); Austin Wilson, Ann Arbor, MI (US); Scott Ijaz, Ann Arbor, MI (US)

(21) Appl. No.: 19/320,398

(22) Filed: Sep. 5, 2025

Related U.S. Application Data
(60) Provisional application No. 63/691,798, filed on Sep. 6, 2024.

(51) Int. Cl. A01K 29/00; A61D 99/00; G01K 13/20; G16H 40/67

(52) U.S. Cl. CPC A01K 29/005; A61D 99/00; G16H 40/67; G01K 13/20

(57) ABSTRACT

A variable sensor system for non-contact vital sign monitoring of animals is described. Non-contact vital sign monitoring may involve correcting measured signals for reflectance and variations in distance between measurements. Non-contact vital sign detection may include temperature detection, and/or measuring cardiovascular and respiratory information. Embodiments are described using a non-contact variable sensor integrated with a weight scale, integrated with a drone, having a pan-tilt monitoring feature, integrated with a kennel, integrated with an automatic pass-fail interface, and including a medication dispensing control module.

### CROSS-REFERENCE TO RELATED APPLICATIONS

This non-provisional patent application is based on and claims priority to U.S. Provisional Patent Application No. 63/691,798, filed on Sep. 6, 2024, the disclosure of which is incorporated by reference as if fully recited herein.

### TECHNICAL FIELD OF THE INVENTION

The present invention relates generally to medical systems, and more particularly to a variable sensor system for non-contact vital sign monitoring of animals. Non-contact vital sign detection may occur by correcting measured signals for reflectance and variations in distance between measurements. Vital sign detection may include detecting a subject's temperature, and/or assessing cardiovascular and respiratory information. In an example embodiment, a variable sensor system comprises a variable radar sensor secured at or proximate to a veterinary weight scale for pets. In another example embodiment, a variable sensor system involves a variable sensor linked to a drone, for example, monitoring individual animals in an animal herd. The sensor of the present invention may include a panning feature for monitoring an animal, may be linked to a kennel or other finite location, may be linked to a pass-fail interface, and may be linked to a medication dispensing control module.

### BACKGROUND AND SUMMARY OF THE INVENTION

Measurement of a subject's physiological health through vital sign monitoring is commonly used, and is useful, in medical diagnostics. Vital signs monitored typically include, for example and without limitation, body temperature, blood pressure, pulse (heart rate), breathing rate (respiratory rate), and blood oxygenation of a subject. To date, these measurements have been accomplished by direct contact with a subject through the use of sensors and hand-held devices. For example, traditionally, veterinarians have tracked the health of animal patients by using various separate direct contact measurement devices (e.g., weight scales, pulse oximeters, thermometers, and the like), and recording measurements from these devices in patient file documents.

An issue with known techniques for tracking the health of animal patients includes that the aforementioned devices may require direct contact with the animal to operate, and the direct contact may make the animal uncomfortable, especially where there are repeated instances of direct contact. Also, in cases where the subject is an animal, it may not be safe to the medical provider to come in contact with or even come close to the animal. Moreover, in cases where vitals are routinely monitored on a moving object (e.g., a moving animal, such as an animal in a herd), the use of contact sensors may be frustrated by the constant movement. In other instances where a subject animal may be violent, or is contaminated with a pathogen (e.g., a virus) or another substance, being close to the subject may pose a health threat to the medical practitioner.

Another issue with known techniques is that it may be time consuming for a veterinarian or other animal health specialist to employ multiple separate measurement devices to gather animal health data. An example of why this may be problematic is that the longer an animal undergoes medical testing, the more uncomfortable or distressed the animal may become. Yet another issue with known techniques is that data organization for the various health metrics being tested may be cumbersome.

A few non-contact vital sign monitoring solutions have been presented, but all fall short of an acceptable solution for various reasons. Some known systems and methods lack accuracy. Known systems and methods also generally fail to account for mobile subjects (e.g., moving animals in a herd), and are generally limited to very particular medical settings (e.g., a veterinary facility). Other existing technologies suffer from the ability to properly detect a subject's vital sign if other subjects are around (e.g., due to the reflected nature of RF technologies). Known systems and methods may suffer from any number of different background noise issues.

The aforementioned shortcomings speak to the need for a system that quickly and remotely obtains and organizes data for a number of different animal health metrics in a variety of settings. In view of this, it is beneficial to have a non-contact variable sensor system for animals, and a corresponding method of obtaining and organizing medical information about an animal involving the non-contact variable sensor system.

According to the present invention in one aspect, an exemplary non-contact variable sensor system for animals involves detecting a subject's vital signs by correcting radar measured signals for reflectance and variations in distance between measurements. One or more computing devices (e.g., including a processor) may execute a sequence of programmed instructions to cause methods described herein to be implemented. Signal scoring methods may be employed, where acquired data and/or information is used for scoring a signal acquired from non-contact vital monitoring. Signal scoring may assist in the accurate measurement of various vitals of a subject, including, for example, subject temperature, and evaluation of subject cardiovascular and respiratory information.

According to the present invention, an exemplary system includes a weight scale configured to detect the weight of an animal, and a variable radar sensor secured at the weight scale. The variable radar sensor may be configured for automatically detecting medically useful information about the animal as the animal is weighed and/or positioned on the weight scale. The system may also include a medical record database containing data about the animal. The system may further include a transmitter configured to transmit data from the weight scale and/or the variable radar sensor to the medical record database. The system may additionally include a processor in association with the weight scale and the variable radar sensor. The processor may be programmed to cause the transmitter to automatically transmit the weight of the animal and the medically useful information to the medical record database to store the weight and the medically useful information in the database.

According to the present invention in another embodiment a non-contact variable sensor is configured to automatically detect medically useful information from an animal's eyes. A processor in association with the non-contact variable sensor may be provided. The processor may be programmed to cause the medically useful information to be stored to the proper animal health record in a database of animal health records maintained in the internet cloud.

Another embodiment of the present invention comprises a drone, a non-contact variable sensor linked to the drone, and a processor, in association with the non-contact variable sensor. The non-contact variable sensor may be configured to automatically detect medically useful information about an animal. The drone may be configured to cause said non-contact variable sensor to be airborne. The non-contact variable sensor may be configured to determine whether the animal is ill. This is especially valuable when monitoring animals outdoors in a herd.

According to the present invention the non-contact variable sensor may be movable via a linear movement member, and/or a rotational movement member. The movement members may be configured to adjust the position of the non-contact variable sensor based on the movement of the animal. The non-contact variable sensor may comprise a camera.

The non-contact variable sensor detected medically useful data about an animal, may include blood pressure information. The processor may be configured to communicate the blood pressure data by way of a wired or wireless network such as a Bluetooth signal. The processor may be configured to communicate the data to an interface of a software application.

The present invention sensor may detect inflammation on an animal and notify a user of a location on the animal where inflammation is detected. A user device may receive the inflammation report and display it on a digital display. The processor may cause the inflammation spot information to be visually represented as a map at the digital display.

The non-contact variable sensor may be positioned at a perimeter of an animal enclosure to automatically detect medically useful information about the animal when the animal is positioned in the enclosure. The non-contact variable sensor may be configured to obtain data for facial recognition of an animal to help identify the animal and the health record for said animal. The enclosure may be a dog kennel. The medically useful information may comprise at least one selected from the group of blood oxygen level, body temperature, and blood pressure.

According to the present invention in other aspects, the non-contact variable sensor may be configured to automatically detect medically useful information about an animal by way of beam steering, phase ray focus (also referred to as phased array focus), or both. The processor may be configured to determine depth information based on information automatically detected by a depth sensing camera and a depth sensing algorithm. The processor, based on information communicated to the processor from the camera, may be configured to determine whether the animal is experiencing problems. The camera may be an RGB imaging camera.

The present invention may be configured with software instructions to cause medically useful information for a particular area of focus of an animal to be displayed at a digital display interface on a remote user's computerized device.

In another embodiment, the non-contact variable sensor system comprises a pass-fail interface, comprising a plurality of indicators, a non-contact variable sensor in electronic communication with the pass-fail interface, and a processor, in association with the non-contact variable sensor. The non-contact variable sensor may be configured to automatically detect medically useful information about an animal in a plurality of animals, as the animal moves past the non-contact variable sensor. The processor may be configured to automatically regulate the plurality of indicators of the pass-fail interface based on the medically useful information when the animal moves past the non-contact variable sensor. The plurality of indicators may include a green light and a red light. The processor may be configured to cause the green light to illuminate when the medically useful information indicates that the animal is healthy, and the processor may be configured to cause the red light to illuminate when the medically useful information indicates that the animal is not healthy.

The non-contact variable sensor together with a death detection module, may be configured to automatically detect whether an animal has died. The processor may be configured to communicate to a user device that an animal has died or that the animal is experiencing problems that may be serious or lead to death.

The processor of the system of the present invention may be configured to regulate medication dispensing of a medication dispensing unit based on the detected medically useful information. For example, the medically useful information may include body temperature information. The processor may be configured to regulate the volume of medication dispensed or the timing of medication dispensed depending upon, for example, whether the animal's body temperature is elevated.

In another embodiment of the present invention, an audio sensor is used with the non-contact variable sensor to automatically detect medically useful information from audible sounds coming from the animal, such as stomach gurgling or respiratory distress.

Advantages of the present invention, may include, promoting comfort of animal patients, reducing effort required of animal health specialists to gather and organize medical information about animal patients, reducing the length of a veterinary appointment, eliminating dangers to animal health specialists related to getting too close to an animal subject, monitoring mobile subjects in a herd, promoting accuracy of animal health data, and the like.

### BRIEF DESCRIPTION OF THE DRAWINGS

Novel features and advantages of the present invention, in addition to those expressly mentioned herein, will become apparent to those skilled in the art from a reading of the following detailed description in conjunction with the accompanying drawings. The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 illustrates a diagrammatic view of a variable sensor system of the present invention involving a weight scale;

FIG. 2 illustrates a variable sensor system of the present invention involving a weight scale in electronic communication with a computer processor;

FIG. 3 illustrates a variable radar sensor scale of the present invention in wireless electronic communication with a computer processor;

FIG. 4 illustrates the exemplary processor of FIG. 2 having a monitor display interface permitting user interaction with an exemplary patient record system;

FIG. 5 illustrates an exemplary patient record system interface of the patient record system of FIG. 4;

FIG. 6 illustrates various exemplary graphs of data stored to the patient record system of FIG. 4;

FIG. 7 illustrates an exemplary variable sensor system of the present invention in electronic communication with a computer processor;

FIG. 8 illustrates an exemplary microchip and an exemplary collar in accordance with a preferred embodiment of the variable sensor of the present invention for use in automatically recognizing a pet to automatically send data gathered by the sensor to the pet's medical record in a database;

FIG. 9 illustrates the exemplary microchip and collar of FIG. 8 in electronic communication with the exemplary variable sensor of FIG. 2B;

FIG. 10 illustrates an exemplary non-contact variable sensor system of the present invention involving a pan-tilt monitoring feature;

FIG. 11 illustrates an exemplary non-contact variable sensor system involving a blood pressure module with Bluetooth signal;

FIG. 12 illustrates an exemplary non-contact variable sensor system of the present invention involving a death detection module;

FIG. 13 illustrates an exemplary non-contact variable sensor system of the present invention involving an audio detection module;

FIG. 14 illustrates a top view of an exemplary drone-integrated non-contact variable sensor of the present invention;

FIG. 15 illustrates an exemplary non-contact variable sensor system of the present invention secured at a pet kennel;

FIG. 16 illustrates an exemplary non-contact variable sensor system of the present invention using beam steering and phase ray focus;

FIG. 17 illustrates exemplary logic for a non-contact variable sensor system of the present invention involving a depth sensing camera;

FIG. 18 illustrates an exemplary non-contact variable sensor system of the present invention including an area focus module;

FIG. 19 illustrates exemplary logic for a non-contact variable sensor system of the present invention involving a detection camera;

FIG. 20 illustrates an exemplary non-contact variable sensor system of the present invention involving a pass-fail interface;

FIG. 21 illustrates an exemplary user software interface for remotely monitoring a non-contact variable sensor system of the present invention;

FIG. 22 further illustrates an exemplary user software interface for the system of FIG. 21;

FIG. 23 illustrates an exemplary user interface for software regulating medication dispensing based on data received from a non-contact variable sensor system of the present invention;

FIG. 24 illustrates exemplary logic for regulating medication dispensing based on measurements of the system of FIG. 23; and

FIG. 25 illustrates an exemplary non-contact variable sensor system of the present invention including an inflammation mode module.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

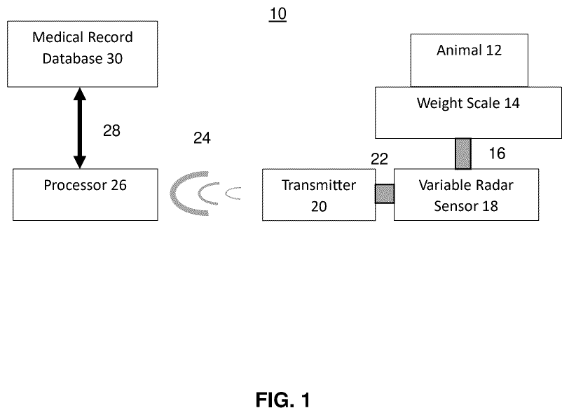

Referring now to FIG. 1, an exemplary non-contact variable radar sensor system for animals 10 comprises a weight scale 14 configured to detect the weight of an animal 12, and a variable radar sensor 18. The variable radar sensor 18 may be configured for automatically detecting medically useful information about the animal 12 as the animal 12 is weighed and/or positioned on the weight scale 14. A processor 26 may be configured to link information about the animal 12 to a medical record database 30 (e.g., programmable by and in communication with 28 the processor) capable of storing and displaying data about various animal patients. A transmitter 20 linked 22 to the variable radar sensor 18 and weight scale 14 may be configured to transmit data from the weight scale 14 and the variable radar sensor 18 to the medical record database 30. The processor 26 may specifically be programmed to cause the transmitter 20 to transmit the identification, weight, and other medically useful information about the animal 12 to the medical record database 30 to store the weight and the other medically useful information in the database 30 (e.g., in a database profile for the animal 12).

The variable radar sensor 18 may be positioned at or proximate to the weight scale 14. For example, the variable radar sensor 18 may be directly connected 16 to the weight scale 14. The variable radar sensor 18, together with the weight scale 14, may provide a check in station for, e.g., automatically detecting the animal's 12 weight, temperature, pulse, respiration rate, and the like when the animal is at a veterinarian appointment. For example, at the beginning of a veterinary appointment, the veterinarian may place the animal 12 on the weight scale 12 to see if there is anything concerning about the animal's current weight, temperature, pulse, and/or respiration rate. An example variable radar sensor component for use with the present invention may be purchased from Seeed Technology Co., Ltd. at the link https://www.seeedstudio.com/60 GHZ-mm Wave-Radar-Sensor-Breathing-and-Heartbeat-Module-p-5305.html. Electronic communication 24 between the transmitter 20 and the processor 26 may involve wired and/or wireless electronic communication channels. The medical record database may involve a software application programmable by any number of different computing devices, as well as electronic storage media. Data may be organized and stored to the medical record database in real time.

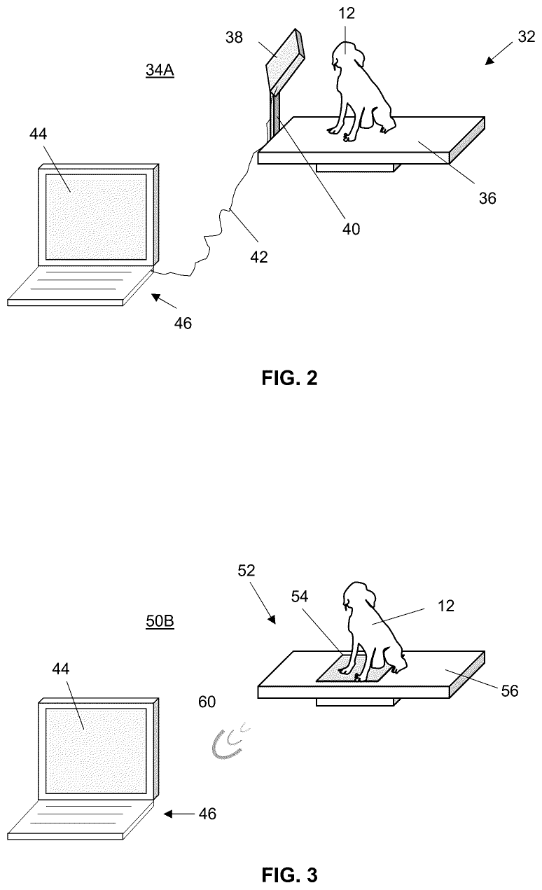

An exemplary system 34A may comprise a variable measurement device 32 including standalone variable radar sensor module 38 positioned proximate to a vet weight scale 36, as shown in FIG. 2. Here, the variable radar sensor module 38 is connected (e.g., by way of clips, fasteners, brackets, clamps, magnets, some combination thereof, or the like) to a support unit 40 (e.g., a rigid support column or pillar made of metal), which may be affixed to the scale 36 such that the module 38 is suspended in close proximity to the animal 12. The positioning of the animal 12 on the scale 36 may permit the scale 36 to transmit measured animal weight to a system processor 46 in communication with a medical record database. Alternatively or additionally, the scale 36 may communicate animal weight to the module 38, and the module 38 may then communicate animal weight along with other medically relevant information about the animal (e.g., measured by one or more sensors of the module 38) to the system processor 46. The weight and other medically relevant information may be displayed 44 on a system interface. The vet weight scale may be a standard digital freestanding veterinary scale, or the like, having the radar/touchless sensor embedded, affixed, attached, secured, within, on, or adjacent thereto. The combination of the sensor together with the scale is an important, beneficial advancement to the veterinary art, in that it uses the clinical weight taking process, when the animal is sitting or standing still, in a position suitable for reasonably close touchless sensor observation, to capture important data without the animal sensing that other data is being collected, simultaneously, in real time, as the animal's weight is being measured. The medical sensor data obtained from the animal and the weight data obtained from the animal on the scale, are then stored in a temporary or permanent database and may be electronically transmitted to a medical chart for the animal, to update a pre-existing chart in the case of a known patient to the veterinarian or establish a first chart for the animal in the case of a new patient. The present invention is a time saver for the animal (less time in the clinical examination setting is less stress on the animal), the animal's owner (multiple types of simultaneous data collection make the clinical exam process go faster), and the veterinarian.

In FIG. 2 embodiment, the variable measurement device 32 is in wired electronic communication (illustrated by wire 42) with the system processor 46. Alternatively, in the FIG. 2 embodiment, the variable measurement device 32 may be in wireless electronic communication with the system processor 46. Any number of different electronic communication channels may be employed without departing from the scope of the present invention. Furthermore, the present invention is not limited to any particular type of scale, radar/touchless sensor device, computing device, and/or database software.

An exemplary system 50B comprising a variable measurement device 52 having an integrated variable radar sensor module 54 positioned in a vet weight scale 56 is shown in FIG. 3. The vet weight scale 56 may be configured with a receptacle capable of housing the variable radar sensor module 54. Alternatively, the variable radar sensor module 54 may be built directly into the scale 56. Any number of techniques may be employed for securing an exemplary variable radar sensor module at or proximate to an exemplary scale. Here, the positioning of the animal 12 on the device 52 may permit the device 52 to transmit measured animal weight, along with other medically relevant information (e.g., measured by module 54) to a system processor 46 in communication with a medical record database. The weight and other medically relevant information may be displayed 44 on a system interface.

In the FIG. 3 embodiment, the variable measurement device 52 may be in wired electronic communication with the system processor 46, or the variable measurement device 52 may be in wireless electronic communication 60 with the system processor 46.

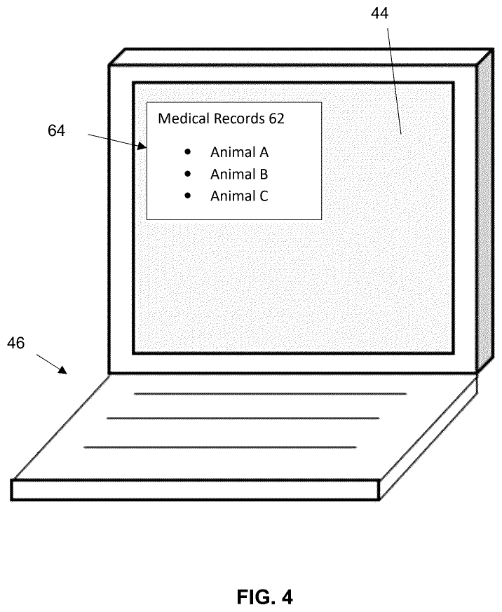

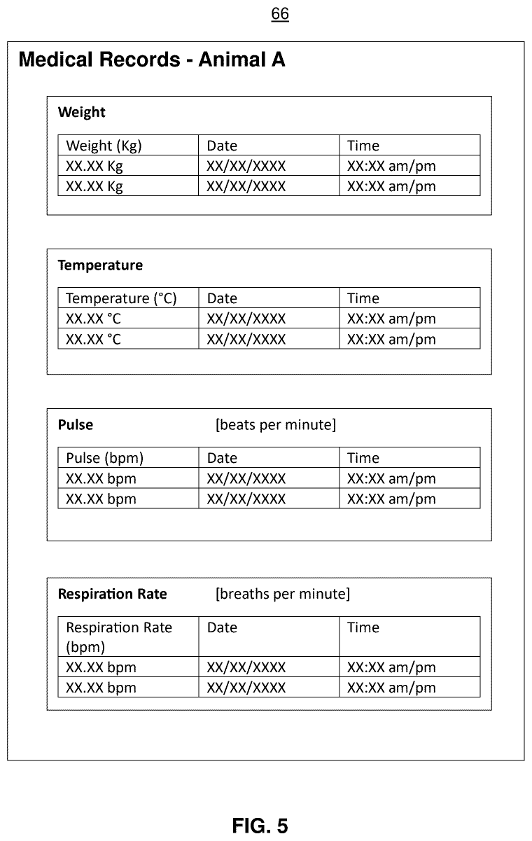

In the FIG. 4 embodiment, an exemplary computing device 46 having a monitor display 44 permits user interaction with an exemplary medical record database (patient record system 62 for various different animals) by way of a system interface 64. In this particular embodiment, a user may interact with the interface 64 to access medically relevant information about the different animals (e.g., Animals A-C) who have been monitored by an exemplary system. Referring to FIGS. 4-5, the user may select one of the animals from the interface 64 to open up a second interface 66 providing various data about the animal.

Referring specifically to FIG. 5, the interface 66 may permit the user to view weight, temperature, pulse, and respiration rate information about the particular animal. The aforementioned information may be stored to the patient record system. Each data point stored to the patient record system may include a date and time label. The user may be permitted to toggle between different unit types (e.g., metric system versus imperial system/U.S. standard) for certain datasets. The various interfaces shown and described herein are merely illustrative, and it will be apparent to those of ordinary skill in the art that any number of different software modules may be employed to promote user interaction with data stored to a medical record database.

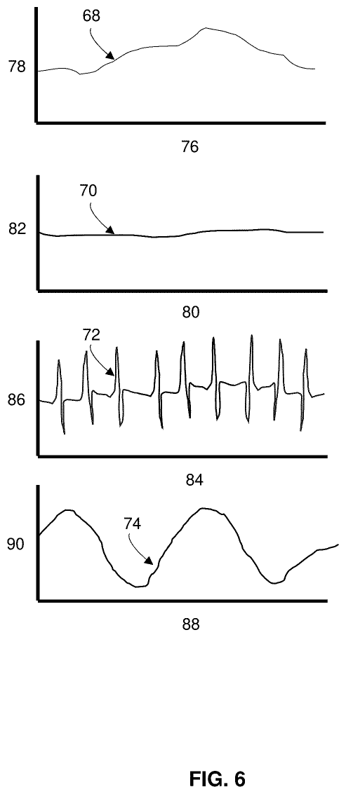

Referring to FIG. 6, various exemplary graphs 68, 70, 72, 74 of data stored to the patient record system of FIG. 4 are shown. Here, the graphs include a weight graph 68, a temperature graph 70, a pulse graph 72, and a respiration rate graph 74. In FIG. 6, the weight graph 68 demonstrates animal weight 78 (e.g., in Kilograms) over a time period 76. The time period 76 may be multiple months or years to reflect animal weight 78 changes over the duration of several appointments. Here, the temperature graph 70 demonstrates animal temperature 82 (e.g., in ° C.) over a time period 80, the pulse graph 72 demonstrates animal pulse 86 (e.g., each wave represents a beat) over a time period 84 (e.g., several seconds), and the respiration rate graph 74 demonstrates animal respiration rate 90 (e.g., each wave represents a breath) over a time period 88 (e.g., several seconds or minutes). Referring to FIGS. 4-6, a user may cause an exemplary graph (e.g., 68, 70, 72, 74) to be displayed by selecting a health metric from a patient record system interface (e.g., 66 displayed on monitor 44). An exemplary system software application may permit a user to make adjustments to the amount of data displayed and the way by which data is displayed.

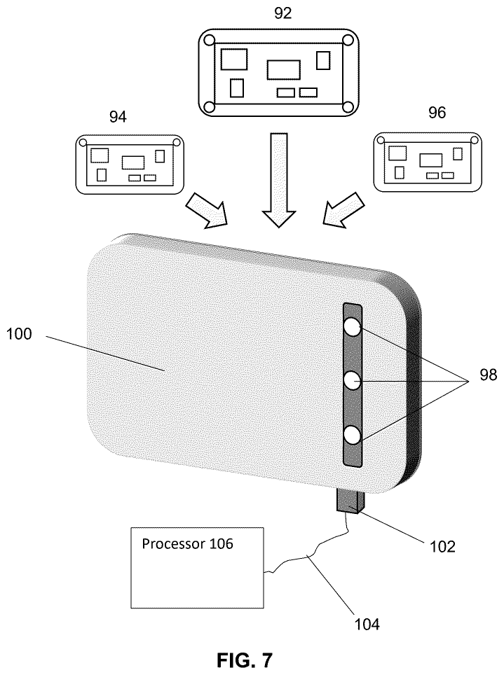

Referring to FIG. 7, an exemplary variable sensor module 100 is shown. The module 100 may include one or more sensor radar frequency output transducers (e.g., 92, 94, 96). Each transducer (e.g., 92, 94, 96) may comprise a circuit board configured to promote transmission and/or receival of module radar signals at any number of different frequencies. A separate sensor radar frequency output transducer may be provided for certain individual health metrics, although such is not required. An exemplary transducer may be adapted to operate over long periods of time in any number of different environments of variable temperature and/or moisture content. Various sensors 98 (e.g., including radar sensors) may be positioned on an external face of the module 100. Modulated radar signals may be registered by the module 100 and transmitted by way of a transmitter to the processor 106, which may organize information from the transmitter as data points. The processor 106 may be in wired electronic communication with the module 100. For example, a wire 104 may extend from the processor 106 and may be received by a data port 102 of the module 100. In alternative embodiments, the processor 106 and the module 100 may be in wireless electronic communication with one another. The processor 106 may be configured to execute a sequence of programmed instructions that cause the processor to implement methods disclosed herein. Data may be collected from one or more sensors mounted to one or more platforms of the variable sensor device 100 or enclosed within one or more housings of the device 100.

The device 100 may be configured to properly detect the vital signs (e.g., heart rate/pulse, blood pressure, body temperature, breathing rate/respiratory rate, blood oxygenation, and the like) of a subject (an animal or human patient having one or more vital signs that may be measured). The device 100 may achieve this by correcting radar measured signals for reflectance and variations in distance between measurements. Furthermore, distance data may originate from sensors such as Light Detection and Ranging (LIDAR) sensors. Sensor device 100 data may be obtained from radiofrequency (RF) signal detection and/or reflection and signal adjudication, thermal imaging, infrared sensors, image capture and analysis, gyroscopes, accelerometers, another sensor, some combination thereof, or the like.

An exemplary system may employ signal scoring methods (e.g., as described in detail in U.S. Pat. No. 11,813,043). Acquired data and/or information may be used for scoring a signal acquired from non-contact vital monitoring, assisting in the accurate measurement of various vitals of a subject, including, without limitation, a subject's temperature, cardiovascular information and respiratory information. Vital signal measurement may also be improved as a result of using multiple measurement techniques to determine the same vital sign being monitored, and to filter and process the information to correct for movement and reflectance from one measurement interval to another, which may occur naturally by the movement of a subject.

An exemplary non-contact vital sign monitoring system having a variable sensor device 100 may include a radio frequency (RF) system at the device 100. The RF system may include one or more RF transmitters, and at least one RF receiver. The RF system (particularly, the RF transmitter and RF receiver) may be used to acquire one or more reflected RF signals from a subject. Doppler shifts in one or more ranges of acquired data may be determined by comparing one iteration of signal acquisition to an adjacent iteration (e.g., using processor 106). The analysis of the reflected RF signals may be used to adjudicate certain vital signs of a subject, including heart rate and respiratory rate (e.g., such as described in U.S. Pat. Nos. 7,903,020, 7,848,896, 8,721,554, 8,814,805, 9,200,945, 11,813,043, and U.S. Pat. Pub. No. US20100204587A1).

Heart rate may be determined by the variable sensor module 100 by, for example, the module 100 measuring a slight change in tissue thickness associated with each systolic phase of a subject's cardiac cycle. Isolated metal conductive tabs may be provided for a subject (e.g., an animal) to stand on. The tabs may contact a sub plate (e.g., similar to the way keys on a computer keyboard contact a subsurface). A location may be sensed through a grid matrix, and an algorithm may permit, for example, the location of each forelimb and the right hindlimb of an animal to be determined. An immediate route for capturing an electrocardiogram (ECG) may be established through the normal Eindhoven Triangle (used conventionally for ECG). Pulse and abnormal cardiac conduction may be readable, allowing remote diagnostic assistance to occur via a connection with a web-based AI program.

Respiration rate may be measured by the variable sensor module 100 by, for example, the module 100 measuring slight changes in diameter of the thorax which naturally occurs with respiration. An exemplary non-contact variable sensor system may be combined with a Brisby system to, for example, improve the accuracy of heart rate and respiration rate data.

It is appreciated that in some environments, RF signal acquisition may be compromised by reflectance or movement. To improve vital sign data acquisition, other data acquisition systems may be provided. For example, an audio/imaging system may be incorporated into the non-contact variable sensor system. The audio/imaging system may include at least one audio/imaging detection device used to acquire information about a subject to resolve vital sign information of that subject. Various audio/imaging detection devices are known in the art, and nothing herein is intended to limit the number of or selection of audio and image detection devices made part of an exemplary audio/imaging system. An audio/image detection device used in an audio/image system may be a video camera with a microphone used to acquire audio and video information of a subject being monitored. Alternatively, or additionally, an image capture system may involve analysis of different wavelengths of light that are not visible light.

Audio and imaging information may be analyzed using one or more computing methods to resolve respiration, heart rate, or other measured vital signs, and may be compared (e.g., using one or more algorithms) to the vital sign information adjudicated through RF reflectance. Traditional signal and image processing techniques, and/or machine learning techniques (e.g., deep learning) may be incorporated into computing methods in order to resolve one or more desired vital signs. An imaging system of the device 100 may be used to detect whether a subject is in view of the device 100. When a subject is not viewable, for example, in a frame of a camera of the device 100, an audio system of the device 100 may be used to determine if the subject is detectable.

The non-contact variable sensor system involving device 100 may further include a thermal detection system. The thermal detection system may be intended to determine thermal properties of a subject, including non-contact monitoring of a subject's body temperature. Infrared filters and/or other filters may be used with exemplary audio and/or imaging systems to assist in vital sign adjudication (e.g., temperature detection). One or more algorithms may be implemented as part of the thermal detection system to compare and improve data obtained by the thermal detection system. The thermal detection system may be used to determine if an animal or other subject has a fever. Additionally, or alternatively, the thermal detection system may be used to determine if an animal or other subject is present proximate to the device 100 (e.g., by comparing the temperature of the subject to the temperature of the subject's surroundings).

Additionally, or alternatively, temperature may be measured by the variable sensor module 100 by way of multiple cameras. For example, the variable sensor module 100 may include a RGB model camera and a thermal imaging camera, and data from an RGB image may be overlaid to a thermal image to permit a processor to evaluate thermograms in an overlaid image to determine temperature of relevant features of an animal, as described in more detail below. Any type and/or number of different sensors (and data acquisition or comparison algorithms related to the sensors) may be provided to analyze subject vitals and/or physiological signals without departing from the scope of the present invention.

An exemplary non-contact variable sensor system may involve signal refinement. Signal refinement may involve at least one gyroscope or accelerometer. Data acquired from an accelerometer and/or gyroscope may be used to calculate the angle of the device 100 with respect to a subject. Based on this, signal may be adjusted to account for angular shifts. An exemplary non-contact variable sensor system may also involve a distance sensor. The distance sensor may be configured to determine straight-line distance between a subject and the device 100 (e.g., to determine if vitals of the subject are capable of being detected). Acceptable distance sensors may utilize any distance adjudication sensor or method known in the art including, for example, Light Detection and Ranging (LiDAR) distance sensors, acoustic distance sensors, infrared (IR) distance sensors, video or RF distance sensors, and the like.

The various systems and components of device 100 may individually and/or collectively communicate with a computing system (e.g., involving processor 106). Different computing devices may be dedicated to different methods of measurement, although such is not required. A computing system may be configured to operate in parallel or sequentially in regard to reading from various sensors. Collected data or information from the device 100 may be directed to the processor 106 for additional processing and/or application of one or more algorithms to be applied on the data/information. To promote real-time display of data measured by the device 100, computing devices linked to and/or integrated with the device 100 may be in electronic communication with one or more electronic display screens. An exemplary electronic display may display measured, resolved, and/or adjudicated vital sign data. A computer readable medium may be provided to store an executable sequence of steps, that when executed performs the desired combination of steps. For example, a CPU may be provided to execute a sequential workflow, a GPU may be provided to execute a parallel computing workflow, an FPGA or ASIC may be implemented in hardware, or a hybrid approach such as FPGA+CPU hardware-software co-design implementation.

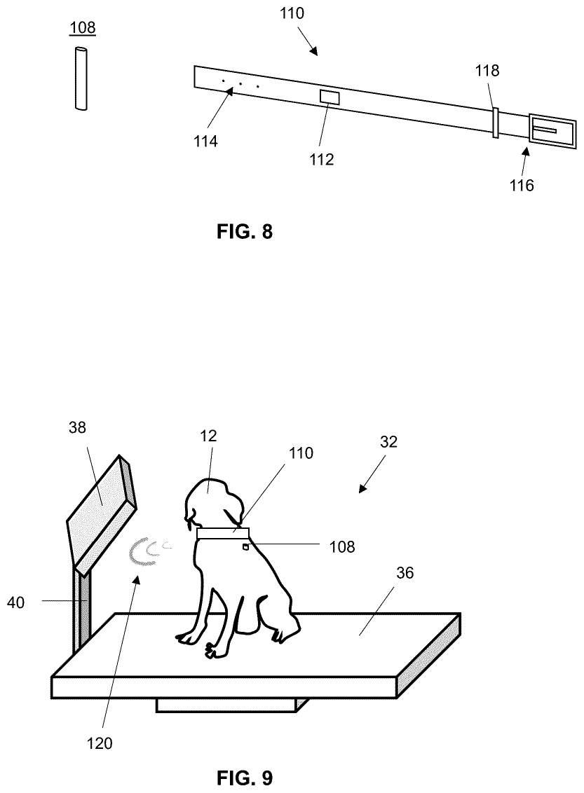

FIG. 8 shows an exemplary microchip 108 and an exemplary ID collar 110. The microchip 108 may be a standard pet microchip. The microchip 108 may be configured to store data about the animal, such as, by way of example and not limitation, the animal's name, the owner's name, the owner's address, the owner's contact information, and the like, and permit said data to be transmitted to and/or downloaded to a medical record database. The microchip 108 may be detected by a scanner providing communication between the microchip 108 and the medical record database. The scanner may be built directly into an exemplary variable sensor module. The scanner may transmit a radar signal to the microchip 108, the radar signal may be modulated by the microchip 108, and then read by the scanner to permit the scanner to transmit information stored to the microchip 108 to the medical record database. Additionally, or alternatively, the ID collar may include a microchip configured to be read by the scanner. The ID collar may include a standard buckle (e.g., punch holes 114, buckle 116, and strap loop 118), although it will be apparent to those of ordinary skill in the art that any number of different collars may be employed without departing from the scope of the present invention.

Referring to FIG. 9, the ID collar 110 and/or microchip 108 may be in electronic communication 120 with an exemplary variable radar sensor module 38 of an exemplary variable measurement device 32, which may be in electronic communication with a processor (not shown). The microchip 108 may be injected into skin proximate to the animal's 12 neck by way of a syringe. The ID collar 110 and/or microchip 108 may provide positive identification of the animal 12 to system software. Referring to FIGS. 8-9, one or more sensors 112 of the ID collar (or a similar device configured to be connected to the animal 12) may measure certain medically useful information about the animal 12 (e.g., animal temperature). In this exemplary embodiment, the variable radar sensor module 38 may be supported above the scale 36 that the animal 12 is positioned on, by a support column 40. The system may be directed to measure various health information about the animal 12 while the animal 12 is positioned on the scale 36. System software may direct measurements to be assigned to datasets specific to the animal 12 based on the identification of the animal provided by the ID collar 110 and/or microchip 108.

Referring back to FIG. 7, the non-contact variable sensor device 100 may include a processor 106 and/or may be in electronic communication with a processor configured to implement software promoting various device 100 functions. The software may be implemented using MATLAB, JAVA, CGI script, Python, some combination thereof, or the like. System software may be stored on an electronic storage medium, and may be executed with the cooperation of a controller and memory. The processor 106 may be provided separate from the device 100, or may be integrated with the device 100. The device 100 may be positioned over or otherwise proximate to a stall (e.g., a horse stall), crate, or kennel. A transducer may cause transmission and/or receiving of device signals. The information communicated to the processor may be stored in the cloud (e.g., so that the information may be accessed by a computing device at any number of different locations). Information about a subject (e.g., a subject's eyes) stored to the cloud may be communicated to the device 100, an electronic display thereof, or the like. The device 100 may be placed in the range of internet connection to ensure information from the cloud (e.g., about a subject's eyes) may be communicated to the device 100, and information from the device 100 (e.g., about a subject's eyes) may be communicated to the cloud. Data acquisition and processing related to the subject may be improved upon by artificial intelligence/machine learning. Data about the subject may be displayed on an electronic display position at the device 100 and/or in electronic communication with the device 100.

Aspects of an exemplary system may be communicated and/or displayed to system users and/or administrators by way of any number of different computer readable mediums implemented according to one or more software modules. Software instructions of an exemplary system may be executed by the processor 106. The present invention is not limited to any particular computing and/or display device, nor is it limited to any particular shape, size, component arrangement and/or design. The present invention is further not limited to the use of an exemplary cloud module with only eye information. An exemplary cloud module may be used to store and communicate information about any number of different features of a subject.

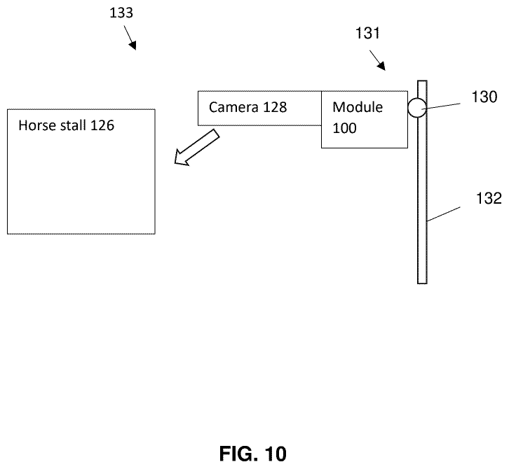

Referring now to FIG. 10, an exemplary non-contact variable sensor system 133 may involve a variable sensor module 100 linked to a panning feature 131 for monitoring an animal. The variable sensor module 100 and pan-tilt monitoring feature 131 may be configured to be positioned at or proximate to a horse stall 126 (e.g., the feature 131 may be hung inside a horse stall). A camera 128 may be integrated to the variable sensor module 100. The pan-tilt monitoring feature may involve a vertical movement module 132, a horizontal movement module (not shown), and an angle adjustment module 130. The camera 128 may track the position of an animal (e.g., a horse) in a stall. The camera 128 may be in electronic communication with the pan-tilt monitoring feature 131, and may cause the pan-tilt monitoring feature 131 to reposition the variable sensor module 100 based on movement of the animal so that the variable sensor module 100 is substantially angled towards and/or approximately aligned with the animal. The angling towards and/or alignment with of the sensor module 100 with the animal may provide for better data acquisition and/or tracking of the animal.

As a non-limiting example, when the animal sits down or otherwise positions itself lower in the stall 126, the sensor module 100 may be rotated down (by the angle adjustment module 130) towards the animal. As another non-limiting example, when the animal sits down or otherwise positions itself lower in the stall 126, the sensor module 100 may be moved downwards linearly (by the vertical movement module 132). As yet another non-limiting example, when the animal moves from a left side of the stall 126 to a right side of the stall 126, the sensor module 100 may be moved horizontally to the right (by the horizontal movement module), and/or rotated to the right (by the angle adjustment module 130).

The vertical movement module 132 may involve a vertically oriented apparatus, and a computer-controlled member connected to the sensor module 100 and vertically oriented apparatus. The member may be configured for automatic movement up and down the vertically oriented apparatus based on feedback from the camera 128 and/or instructions of the sensor module 100. The horizontal movement module may involve a horizontally oriented apparatus, and a computer-controlled member connected to the sensor module 100 and horizontally oriented apparatus. The member may be configured for automatic movement side to side along the horizontally oriented apparatus based on feedback from the camera 128 and/or instructions of the sensor module 100. The angle adjustment module 130 may involve a computer-controlled hinge and/or joint connected to the sensor module 100, and configured to cause rotation of the sensor module 100 based on feedback from the camera 128 and/or instructions of the sensor module 100.

Aspects of the system 133 may be communicated and/or displayed to system users and/or administrators by way of any number of different computer readable mediums. Aspects of the system 133 may be implemented according to one or more software modules of one or more processors. Software instructions of the system 133 may be executed by the one or more processors. It will be apparent to one of ordinary skill in the art that the system 133 is not limited to use with an animal stall, nor is it limited to use with horses.

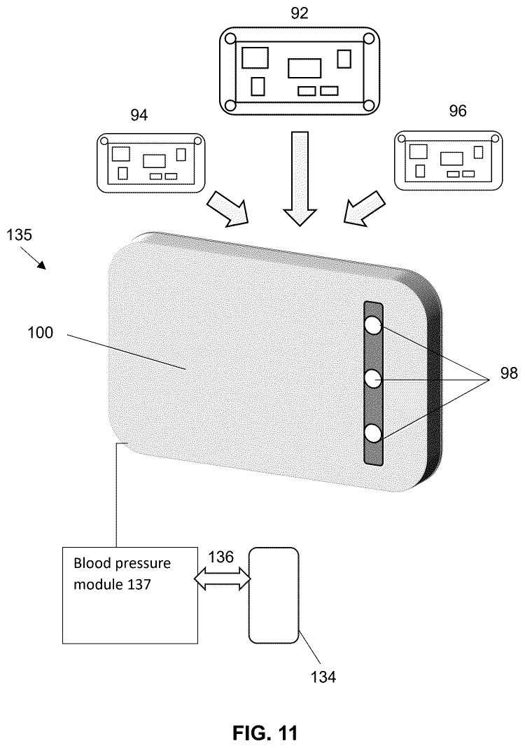

Referring now to FIG. 11, an exemplary non-contact variable sensor system 135 involving a blood pressure module 137 with Bluetooth signal 136 is shown. In this particular embodiment, a non-contact variable sensor device 100 includes one or more sensors 98 (e.g., radar sensors and/or other RF sensors) configured to obtain information about a subject's blood pressure. A transducer (e.g., 92, 94, 96) may cause transmission and/or receival of device signals. A processor may be provided separate from the device 100, and/or may be integrated with the device 100. The sensors 98 may include one or more RF transmitters, and at least one RF receiver, which may be used to acquire one or more reflected RF signals from a subject. Doppler shifts in one or more ranges of acquired data may be determined by comparing one iteration of signal acquisition to an adjacent iteration (e.g., using the processor). The analysis of the reflected RF signals may be used to adjudicate certain vital signs of a subject, including blood pressure.

A blood pressure module 137 (implemented based on software instructions of the processor) may determine subject blood pressure over time. Subject blood pressure data may be communicated to a user device 134 by way of a Bluetooth signal 136. As a non-limiting example, an animal at a veterinarian appointment may be positioned proximate to the device 100. The sensors 98 of the device 100 may be used with the blood pressure module 137 to adjudicate blood pressure of the animal. The processor may be configured to communicate the blood pressure information by way of a Bluetooth signal to an interface of a software application. For example, the veterinarian (and/or the animal's owner) may view, by way of a software application user interface viewable on the user device 134 (e.g., a smart device or other computing device having an electronic display), the animal's blood pressure.

The communication of the animal's blood pressure from the blood pressure module 137 to the user device 134 may occur wirelessly, such as by way of a Bluetooth signal. One or more algorithms of the blood pressure module 137 may be configured to promote blood pressure data acquisition, refinement, and or communication. A software application configured with instructions to receive (e.g., by Bluetooth signal) and display blood pressure data may be executed by the user device 134. A complimentary download of the software application may be provided to users of the system 135. An exemplary software application is not limited to use with blood pressure data, and may be used to refine, communicate and/or view data pertaining to any number of different subject vitals.

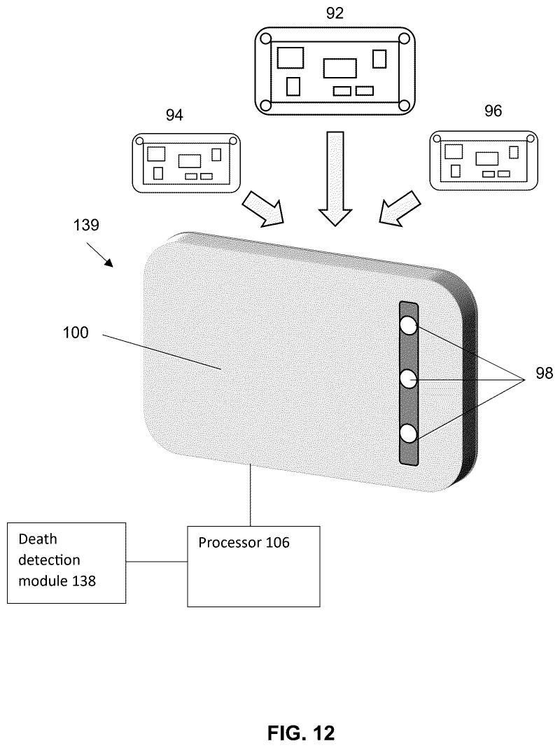

Referring now to FIG. 12, an exemplary non-contact variable sensor system 139 involving a death detection module 138 is shown. In this particular embodiment, a non-contact variable sensor device 100 includes one or more sensors 98 (RF sensors, cameras, audio recording devices, some combination thereof, or the like) configured to evaluate whether a subject is alive or not. A transducer (e.g., 92, 94, 96) may cause transmission and/or receival of device signals. A processor 106 may be provided separate from the device 100, and/or may be integrated with the device 100. The processor 106 may be configured with software instructions to implement the death detection module 138. The death detection module 138 may be configured to, based on data communicated from the sensors 98, determine when a subject (e.g., an animal in a herd) has died, and may communicate that the subject has died to a user device (e.g., an animal owner's smart phone).

As a non-limiting example, at least one sensor of the sensors 98 may comprise a camera configured to track motion of an animal. The death detection module 138 may be in electronic communication with the camera. Where the death detection module 138 has registered, by way of the camera, that a lack of motion over an extended amount of time has occurred for the animal, the death detection module 138 may conclude and communicate to a user (e.g., by way of a digital message to a user device) that the animal has died.

As another non-limiting example, the sensors 98 may include one or more RF transmitters, and at least one RF receiver configured to acquire one or more reflected RF signals from an animal. The sensors 98 may be in electronic communication with the death detection module 138. Doppler shifts in one or more ranges of acquired data may be determined by comparing one iteration of signal acquisition to an adjacent iteration (e.g., using the processor 106). The analysis of the reflected RF signals may be used to adjudicate certain vital signs of a subject, including heart rate and respiratory rate. Where a lack of vital sign feedback is registered (e.g., where heart rate and/or respiratory rate is zero), the death detection module 138 may conclude and communicate to a user (e.g., by way of a digital message to a user device) that the animal has died.

As yet another non-limiting example, the device 100 may include a temperature detection module in electronic communication with the death detection module 138. Where a cold temperature is detected for an animal, the death detection module 138 may conclude and communicate to a user that the animal has died. An exemplary non-contact variable sensor system involving a death detection module is not limited to any particular sensing mechanism for determining that an animal has died.

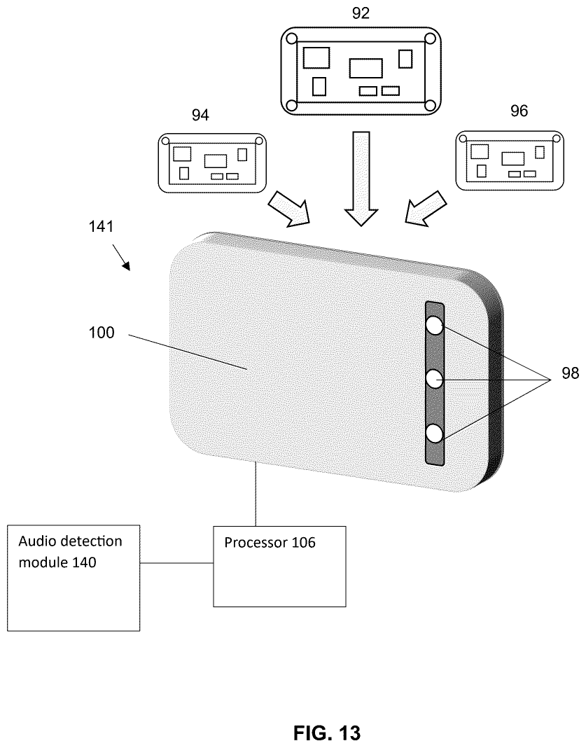

Referring now to FIG. 13, an exemplary non-contact variable sensor system 141 involving an audio sensor module 140 is shown. In this particular embodiment, a non-contact variable sensor device 100 includes one or more sensors 98 (RF sensors, cameras, audio recording devices, some combination thereof, or the like) configured to evaluate whether a subject (e.g., a horse) is experiencing audible distress, such as stomach gurgling or respiratory distress. A transducer (e.g., 92, 94, 96) may cause transmission and/or receival of device signals. A processor 106 may be provided separate from the device 100, and/or may be integrated with the device 100. The processor 106 may be configured with software instructions to implement the stomach gurgling detection module 140. The audio detection module 140 may be configured to, based on data communicated from the sensors 98, determine when a subject (e.g., a horse proximate to device 100) is experiencing audible signs of distress such as loud stomach gurgling. Where, for example, a horse is experiencing stomach gurgling, a veterinarian may receive a message at a user device that the horse is experiencing stomach gurgling, and the veterinarian may diagnose the horse with colic. The veterinarian may then treat the horse accordingly.

As a non-limiting example, at least one sensor of the sensors 98 may comprise an audio recording device configured to track sound being emitted from a horse's digestive track. The stomach gurgling detection module 140 may be in electronic communication with the audio recording device. Where the stomach gurgling detection module 140 has registered, by way of the audio recording device, that atypical sound consistent with stomach gurgling is occurring over an extended amount of time, the stomach gurgling detection module 140 may conclude and communicate to a user that the horse is experiencing stomach gurgling.

As another non-limiting example, the sensors 98 may include one or more RF transmitters, and at least one RF receiver configured to acquire one or more reflected RF signals from an animal. The sensors 98 may be in electronic communication with the stomach gurgling detection module 140. Doppler shifts in one or more ranges of acquired data may be determined by comparing one iteration of signal acquisition to an adjacent iteration (e.g., using the processor 106). The analysis of the reflected RF signals may be used to adjudicate stomach gurgling. An exemplary non-contact variable sensor system involving a stomach gurgling detection module is not limited to any particular sensing mechanism for determining that an animal is experiencing stomach gurgling.

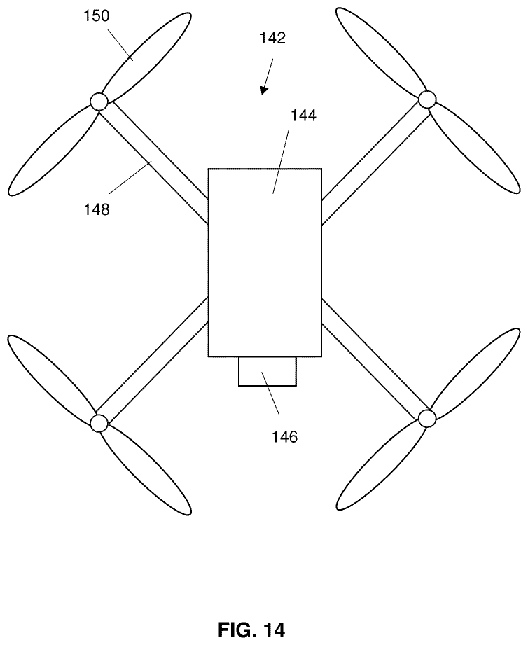

Referring now to FIG. 14, an exemplary non-contact variable sensor system 142 including a drone-integrated sensing device 144 is shown. The drone-integrated sensing device 144 may include or at least be in communication with a processor configured to execute software instructions for one or more software modules for registering, refining, communicating and/or displaying (e.g., on a smart device display screen) animal vital sign data. The drone-integrated sensing device 144 may be substantially similar to the device 100 of FIGS. 7 and 10-13. The drone-integrated sensing device 144 may include at least one camera 146 in electronic communication with the processor. The at least one camera 146 may be configured to track movement (or lack thereof) of animals in a herd. The at least one camera 146 may be configured to permit temperature detection of individual animals in a herd of animals to be evaluated.

The drone-integrated sensing device 144 may include one or more RF transmitters, and at least one RF receiver configured to acquire one or more reflected RF signals from animals in a herd. The sensors may be in electronic communication with the processor. Doppler shifts in one or more ranges of acquired data may be determined by comparing one iteration of signal acquisition to an adjacent iteration (e.g., using the processor). The analysis of the reflected RF signals may be used to adjudicate one or more vital signs or other health aspects of animals in a herd.

The drone-integrated sensing device 144 may be centrally positioned at a drone body between a plurality of arms 148 each linked to a propeller 150. The propellers 150 may be configured to cause and regulate lift and thrust of the system 142, so that the system 142 may be airborne (e.g., over a herd). A flight controller (not shown) may be centrally positioned above or otherwise proximate to the drone-integrated sensing device 144. The flight controller may be configured to cause motion of the propellers 150 (by way of motors and axles regulated by the flight controller). The flight controller may be linked to an antenna and/or control receiver (not shown), which may be configured to transmit flight instructions from a user controller (e.g., a radio controller) (not shown) to the flight controller. Alternatively, or additionally, the antenna and/or control receiver may transmit flight instructions from a processor configured to automatically assign a flight path to the system 142. The system 142 may be powered by one or more batteries, which are preferably rechargeable.

The system 142 may be advantageous for monitoring health aspects of various individual cows and/or horses in a herd located in (and potentially spread out over) a large tract of land. For example, where a sick animal is identified (e.g., by way of identifying elevated temperature in the animal) in the herd, the owner of the herd may remove that animal from the herd to prevent other animals from contact with the sick animal. An exemplary non-contact variable sensor system including a drone-integrated sensing device is not limited to any particular type, dimensions, and/or category of drone, nor is it limited to any particular sensing mechanism for evaluating health aspects of animals below the drone.

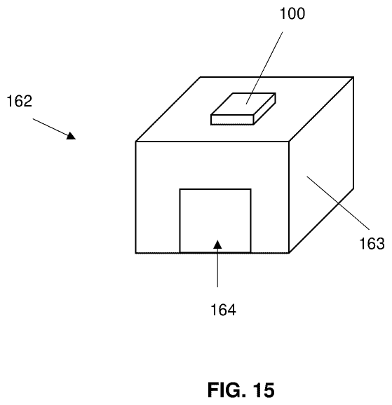

Referring now to FIG. 15, an exemplary non-contact variable sensor system 162 involving a kennel 163 having a non-contact variable sensing device 100 linked thereto is shown. The device 100 may be positioned at or near the perimeter of a kennel, cage, or other compartment/enclosure where an animal may be positioned in (e.g., through an opening 164) and restricted to (e.g., for transportation of the animal). The sensing device 100 may include or at least be in electronic communication with one or more processors configured to execute software instructions for one or more software modules for registering, refining, communicating and/or displaying (e.g., on a smart device display screen) animal vital sign or other health aspect data. The sensing device 100 may include one or more radar sensors, audio sensors, camera sensors, some combination thereof, or the like. One or more sensors of the sensing device 100 may be configured to measure vital sign information and/or other health aspects of a subject.

For example, the sensing device 100 may be configured to evaluate animal temperature, blood oxygen level, blood pressure, some combination thereof, or the like. As another example, a camera sensor of the device 100 may be configured to capture imagery of an animal's face, and one or more system 162 processors may be configured with instructions to evaluate the animal's mood based on the captured imagery. An exemplary non-contact variable sensor system involving a kennel or other enclosure having a non-contact variable sensing device linked thereto is not limited to any particular type, dimensions, and/or category of enclosure, nor is it limited to any particular sensing mechanism for evaluating health aspects of an animal in the enclosure.

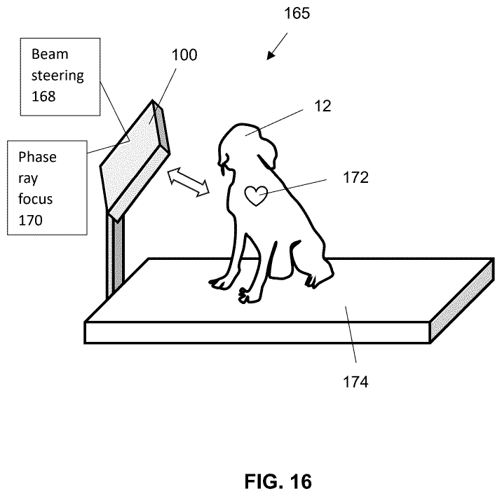

Referring now to FIG. 16, an exemplary non-contact variable sensor system 165 involving a beam steering module 168 and phase ray focus module 170 is shown. Here, a non-contact variable sensing device 100 is configured to detect medically useful information about an animal 12 by way of beam steering, phase ray focus, or both. The non-contact variable sensor device 100 may be positioned proximate to a surface 174 that an animal subject 12 is positioned on. The sensing device 100 may include or at least be in electronic communication with a processor configured to execute software instructions for one or more algorithms of the beam steering module 168 and/or phase ray focus module 170 that promote the collection of data about the subject's heart 172.

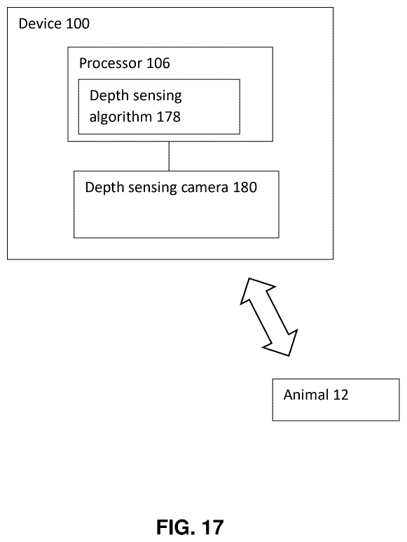

Referring now to FIG. 17, an exemplary non-contact variable sensor device 100 may include a depth sensing camera 180. The depth sensing camera 180 may track an animal 12 (e.g., a dog, cat, pig, chicken, hawk, falcon, horse, or the like), and may communicate to a processor 106 information about the position of the animal 12. The sensing device 100 may include the processor 106, or may at least be in electronic communication with the processor 106. The processor 106 may be configured to determine depth information about the animal 12 based on information automatically detected by the depth sensing camera 180 and a depth sensing algorithm 178. The processor 106 may be configured to execute software instructions for the depth sensing algorithm 178. The depth sensing algorithm 178 may incorporate information about the position of the animal 12 obtained from the depth sensing camera 180. The depth sensing algorithm may determine based on said information the depth of the animal 12 with respect to the device 100.

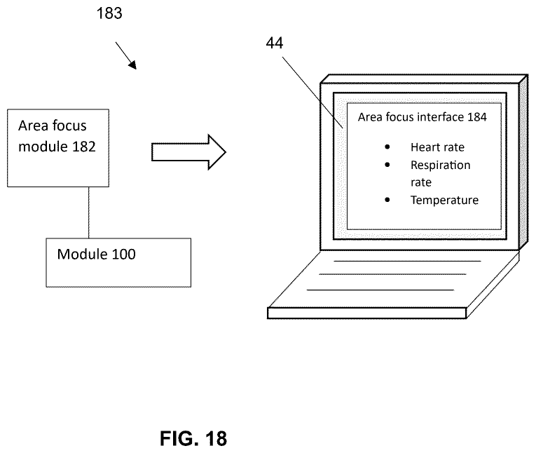

Referring now to FIG. 18, an exemplary non-contact variable sensor system 183 involving an area focus module 182 is shown. In this particular embodiment, information obtained from the sensors (not shown) of a non-contact variable sensor module 100 is communicated to a processor of the system 184. The processor may be configured to execute software instructions for the area focus module 182. The area focus module 182 may be configured to register the information obtained from the sensors, and overlay the information onto a digital display 44 (e.g., the display screen of a smart device or other computing device) with a focus on a particular animal health aspect. An area focus interface 184 of the system 165 may be displayed at a digital display 44. The system 184 may permit a user to display at a digital display interface various different categories of animal health information, including, for example, heart rate, respiration rate, and/or temperature.

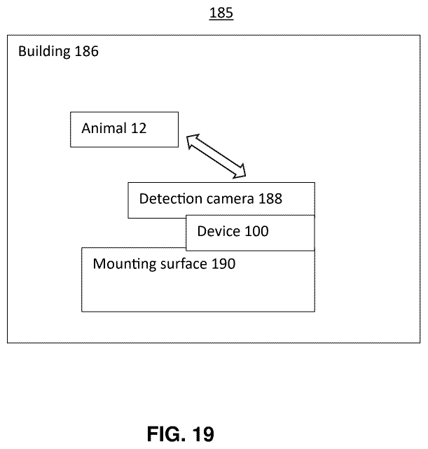

Referring now to FIG. 19, an exemplary non-contact variable sensor system 185 involving a detection camera 188 is shown. A non-contact variable sensing device 100 may include the detection camera 188. The device 100 may be positioned on a mounting surface 190 within a building 186 or other enclosure having an animal 12 therein. The device 100 and/or the detection camera 188 may be configured for linear and/or rotational movement with respect to the mounting surface 190. This may permit the detection camera 188 to track the animal 12. The detection camera 188 may include an RGB imaging camera. Video and/or other imagery from the camera 188 may be communicated to any number of different external user devices (e.g., smart phones, tablets, and/or other computing devices). Said communication may occur in real time.

Furthermore, video and/or other imagery from the camera 188 may be communicated to a health evaluation module. One or more processors may be provided separate from the device 100, and/or may be integrated with the device 100. The processor(s) may be configured with software instructions to implement the health evaluation module. The health evaluation module may be configured to, based on video, other imagery, and/or other data from sensors (e.g., 188) of the device 100, determine where a subject is experiencing illness. For example, when animal diarrhea, vomiting, panting, some combination thereof, or the like is recorded by the detection camera 188 and communicated to the health evaluation module, the health evaluation module may conclude that the animal 12 is sick, and communicate that the animal is sick to a system 185 user. An exemplary non-contact variable sensor system involving a detection camera is not limited to any particular sensing mechanism for evaluating animal health aspects.

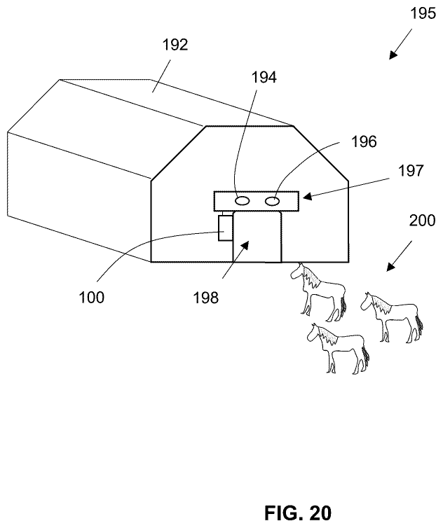

Referring now to FIG. 20, an exemplary non-contact variable sensor system 195 involving a pass-fail interface 197 is shown. In this particular embodiment, an exemplary non-contact variable sensing device 100 is in electronic communication with the pass-fail interface 197. The device 100 may be positioned proximate to the entrance 198 of a barn 192 or other enclosure (e.g., a show arena) configured to house more than one animal (e.g., a group of horses 200). The device 100 may include various sensors for registering, processing, and/or communicating health data for each animal 200. Device 100 sensors may include RF sensors (e.g., an RFID temperature reader, radar and/or other RF sensors for detecting heart rate and/or respiration rate, some combination thereof, or the like), camera sensors, and/or audio sensors. The sensor measurements from device 100 may be communicated to an evaluation module implemented according to software instructions of one or more system 195 processors. System 195 processors may linked to the device 100 and the pass-fail interface 197.

The processor(s) may, based on whether one or more measurements from the sensors exceed a threshold (e.g., an RFID temperature reader measurement exceeds normal body temperature for a horse 200), determine whether an animal 200 passing through an entrance 198 is likely sick. In the embodiment shown, the pass-fail interface 197 includes a first indicator 194 and a second indicator 196. The pass-fail interface may substantially resemble a scoreboard or traffic light. The first indicator 194 may be a green light, and the second indicator 196 may be a red light. The evaluation module may be configured to cause the green light to illuminate when the animal 200 passing through the entrance 198 has measured health aspects consistent with a healthy animal (e.g., normal body temperature). The evaluation module may be configured to cause the red light to illuminate when the animal 200 passing through the entrance 198 has measured health aspects consistent with a sick animal (e.g., elevated body temperature). Where the red light illuminates, an overseer of the group of animals 200 may remove the sick animal from the rest of the group to prevent the spread of illness. An exemplary non-contact variable sensor system involving a pass-fail interface is not limited to any particular sensing mechanism for evaluating animal health aspects.

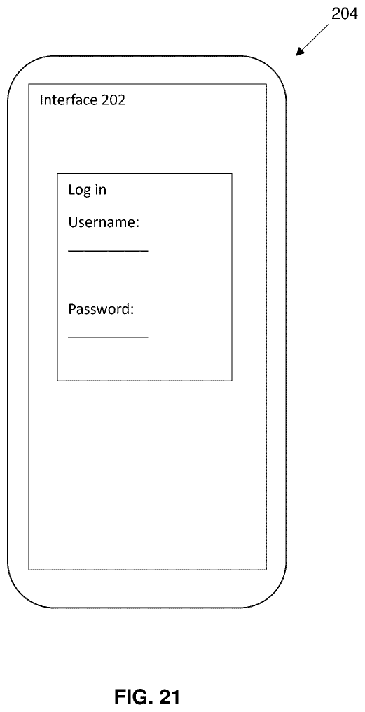

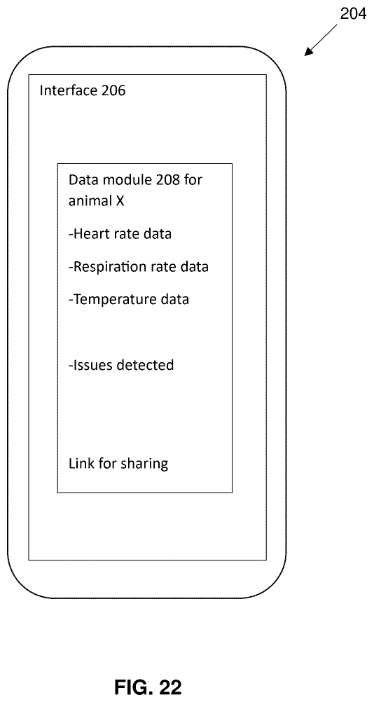

Referring now to FIGS. 21-22, user interfaces 202, 206 for a software application of an exemplary non-contact variable sensor system 204 are shown. A first user interface 202 may be displayed on a digital display of a computing device (e.g., a smart device screen or other computer screen). The first interface 202 may include a log in interface where a user may enter username and/or password information. The user may be granted access to the system 204 software application after entering appropriate username and/or password information. Thereafter, a second user interface 206 may be displayed at the digital display. The second interface 206 may permit the user to view information from and/or interact with a data module 208 for an animal. The information may include various health data measured, refined, and/or communicated from a non-contact variable sensing device (not shown). The health data may include animal heart rate data, respiration rate data, and/or temperature data.

The health data may be displayed at the interface 206 as text, graphs, charts, and/or other visual depictions. A sharing module of the system 204 may permit the user to share various health data viewable at the interface 206 of a user device to other system 204 users. Any number of different system 204 users may be permitted to view and/or interact with the health data. Health data may be shared by way of an electronic link, such as a hyperlink. Health data may include some or all medically useful information detected by an exemplary non-contact variable sensing device.

The system 204 may also be configured to send an alert to the interface 206 when an animal is experiencing significant problems (detected by one or more non-contact variable sensing devices). For example, where a non-contact variable sensing device assesses a health aspect of an animal (e.g., animal body temperature measured by an RFID temperature reader, problematic animal behaviors such as vomiting and/or panting detected by a system 204 camera), and the measurement is determined by a software module of the system 204 to be consistent with an illness or another problem in the animal, the system 204 may employ an automatic message model to send an automated message to the interface 206 of a user device. The automated message may include a link to relevant health data, and/or to video/imagery of the animal. The automated message may indicate to a user that animal X is sick, distressed, and/or is otherwise experiencing problems. Relevant health data shared to a user device may include some or all medically useful information detected by an exemplary non-contact variable sensing device.

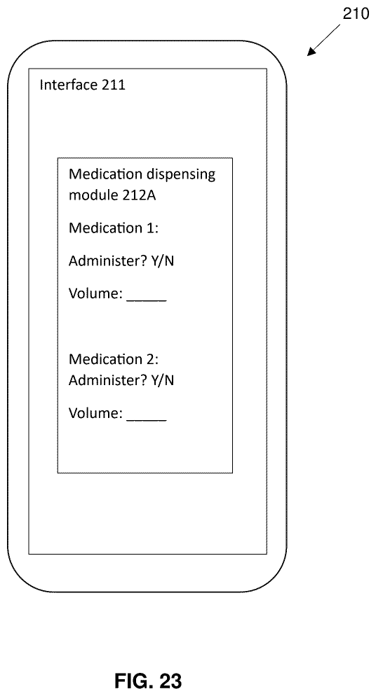

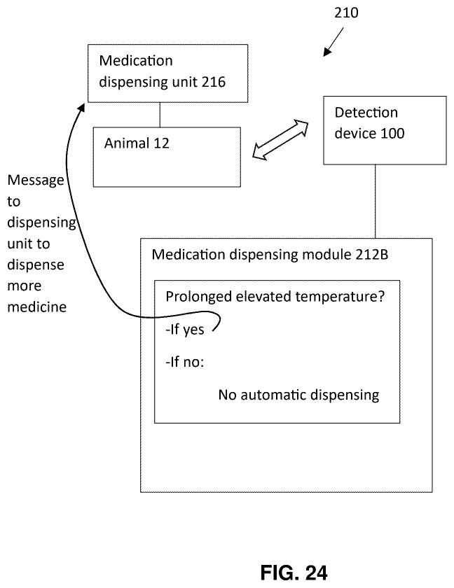

Referring now to FIGS. 23-24, an exemplary non-contact variable sensor system 210 may be configured to regulate medication dispensing based on measurements of the system 210. The system 210 may include a detection device 100 providing one or more sensors for registering, processing, and/or communicating health data for an animal 12. Device 100 sensors may include RF sensors (e.g., an RFID temperature reader, radar and/or other RF sensors for detecting heart rate and/or respiration rate, some combination thereof, or the like), camera sensors, and/or audio sensors. The sensor measurements from device 100 may be communicated to an evaluation module executed according to software instructions of one or more system 210 processors linked to the device 100.

The processor(s) may execute software instructions for a medication dispensing module. The medication dispensing module may, based on whether one or more measurements from the sensors exceed a threshold (e.g., an RFID temperature reader measurement exceeds normal body temperature for the animal 12), evaluate whether medication should be provided to the animal 12. According to one exemplary medication dispensing module 212A, a user interface 211 may be displayed on a digital display of a computing device (e.g., a smart device screen or other computer screen). A user (e.g., a veterinarian) may, based on measurements of the device 100 and/or evaluation of the medication dispensing module, instruct the system 210 to administer one or more medications to an animal, and may assign volumes to the medications being administered. The user interface 211 may be in electronic communication with a medication dispensing unit 216 (e.g., an IV unit).

According to an alternative medication dispensing module 212B, medication may be automatically administered from a medication dispensing unit 216 without direct user input. The medication dispensing module 212B may, based on whether one or more measurements from the sensors of device 100 exceed a threshold (e.g., an RFID temperature reader measurement exceeds normal body temperature for the animal 12), automatically assign instructions to the medication dispensing unit 216. For example, when the medication dispensing module 212B registers that prolonged elevated temperature is occurring with animal 12, the medication dispensing module 212B may communicate to the dispensing unit an instruction to dispense more medicine into the animal (e.g., by way of an IV device, or via a medicine regulator in conjunction with a feed trough). If prolonged elevated temperature is not occurring with animal 12, the medication dispensing module 212B may provide no dispensing instructions to the medication dispensing unit 216.

A veterinarian may predetermine and use a system 210 user interface to assign the amount of medicine to be administered, the type of medicine to be administered, the medication administration and/or health aspect testing intervals, some combination thereof, or the like. For example, the veterinarian may dictate that if after 4 hours, the animal 12 has a normal body temperature, volume A of medicine X will be administered to the animal 12 by the system 210. As another non-limiting example, the veterinarian may determine that if after 4 hours, the animal 12 has an elevated body temperature, volume B of medicine X will be administered to the animal 12 by the system 210, where volume B is greater than volume A. An exemplary non-contact variable sensor system with medication dispensing is not limited to any particular type or number of medication dispensing unit(s), nor is it limited to any particular sensing mechanism for evaluating animal health aspects.

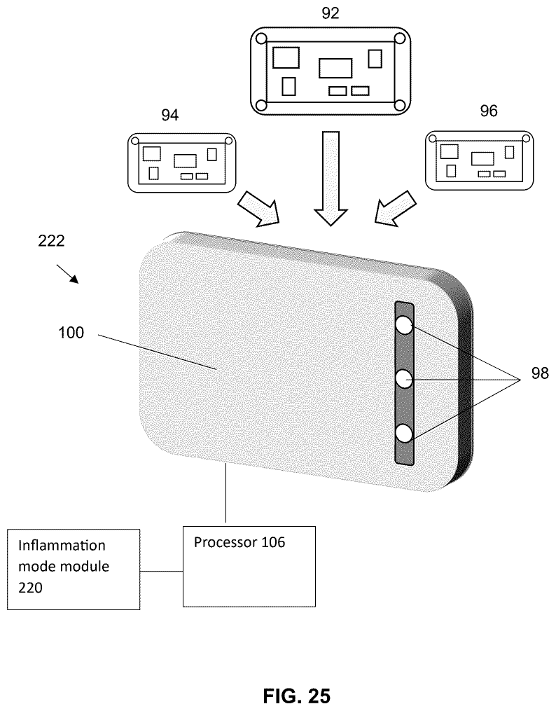

Referring now to FIG. 25, an exemplary non-contact variable sensor system 222 involving an inflammation mode module 220 is shown. In this particular embodiment, a non-contact variable sensor device 100 includes one or more sensors 98 configured to evaluate inflammation in a subject (e.g., an animal). A transducer (e.g., 92, 94, 96) may cause transmission and/or receival of device signals. A processor 106 may be provided separate from the device 100, and/or may be integrated with the device 100. The processor 106 may be configured with software instructions to implement the inflammation mode module 220. The inflammation mode module 220 may be configured to, based on data communicated from the sensors 98, determine where a subject is experiencing inflammation.

At least one sensor of the sensors 98 may comprise an RFID temperature reader. The inflammation mode module 220 may be in electronic communication with the RFID temperature reader. Where the inflammation mode module 220 has registered, by way of the RFID temperature reader, that atypical temperature is occurring at one or more particular locations on an animal, the inflammation mode module 220 may conclude that inflammation is occurring at said particular locations (“hot spots”), and communicate to a user that the animal is experiencing inflammation at the hot spots. A user interface (e.g., an electronic display of a computing device) may provide a map or other visual representation of where the animal is experiencing hot spots. This may assist a veterinarian in treating the animal. An exemplary non-contact variable sensor system involving an inflammation mode module is not limited to any particular sensing mechanism for evaluating inflammation.

Referring to FIGS. 1-25, an exemplary system may include a transmitter configured to transmit data from a non-contact variable sensor to a medical record database. Medically useful information may be detected by an exemplary sensor automatically, and may be communicated to a medical record database automatically (by way of a processor linked to a sensor of a sensing device). Any embodiment described herein may include a processor configured to communicate (e.g., by way of a Bluetooth signal) medically useful information detected by a non-contact variable sensor to an interface of a software application. Any embodiment described herein may be combined with any other embodiment(s) described herein to provide a non-contact variable sensor device and/or system where various different health metrics among one or more subjects, particularly animals, may be tracked.

The exemplary embodiments herein disclosed are not intended to be exhaustive or to unnecessarily limit the scope of the invention. The exemplary embodiments were chosen and described in order to explain the principles of the present invention so that others skilled in the art may practice the invention. Although embodiments described herein were described with reference to obtaining and organizing medical information about an animal, it will be apparent to one of ordinary skill in the art that an exemplary embodiment of the present invention may also be useful for other purposes. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

Any embodiment of the present invention may include any of the features of the other embodiments of the present invention. Furthermore, certain operations described herein may be performed by one or more electronic devices. Each electronic device may comprise one or more processors, electronic storage devices, executable software instructions, and the like configured to perform the operations described herein. The electronic devices may be general purpose computers or specialized computing devices. The electronic devices may comprise personal computers, smartphone, tablets, databases, servers, or the like. The electronic connections and transmissions described herein may be accomplished by wired or wireless means. The computerized hardware, software, components, systems, steps, methods, and/or processes described herein may serve to improve the speed of the computerized hardware, software, systems, steps, methods, and/or processes described herein.

What is claimed is:

1. A medical examination system for animals, comprising:
a non-contact variable sensor configured for automatically detecting medically useful information about an animal, in substantially real time;
a medical record database containing data about said animal;
a transmitter, configured to transmit data from said non-contact variable sensor to said database; and
a processor in association with said non-contact variable sensor, said processor programmed to cause said transmitter to transmit said medically useful information to said medical record database to store said medically useful information in said medical record database for said subject animal.

2. The system of claim 1, wherein said sensor is configured to detect a microchip physically associated with said animal to particularly identify said animal.

3. The system of claim 2, wherein said microchip is secured within an animal collar.

4. The system of claim 1, wherein said medically useful information includes a pulse, temperature and respiration rate of said animal.

5. The system of claim 2, wherein said medically useful information is assigned to an electronic record in a database, specific to said animal, based on identification information about said animal provided by said microchip.

6. The system of claim 1, wherein said sensor is secured at an animal weight scale, and is adapted to detect said medically useful information while said animal is being weighed.

7. The system of claim 1, wherein said transmittal of medically useful information comprises a Bluetooth wireless communication network.

8. The system of claim 1, further including a camera and facial recognition software, to enable said system to identify an animal and to send collected medically useful information about said animal to said database.

9. The system of claim 1, further comprising a camera and associated inflammation detection software for detecting an inflammation zone on said animal.

10. A medical examination system for animals, comprising:
a non-contact variable sensor movably secured at a finite location, said non-contact variable sensor configured for automatically detecting medically useful information about an animal;
an electronic medical record database containing data about said animal;
a transmitter, configured to transmit data from said non-contact variable sensor to said database;
a processor in association with said non-contact variable sensor, said processor programmed to cause said transmitter to transmit said medically useful information to said medical record database to store said medically useful information in said medical record database for a subject animal; and
a movement member in physical association with said sensor to automatically move said sensor in the direction of said animal as said animal moves about said finite location.

11. The system of claim 10, wherein said finite location is an animal enclosure and said non-contact variable sensor is positioned at a perimeter of said animal enclosure to automatically detect medically useful information about said animal when said animal is contained within said enclosure.

12. The system of claim 11, wherein said enclosure is a horse stall.

13. The system of claim 1, wherein said sensor is configured to detect said medically useful information by at least one of beam steering and phase ray focus.

14. A medical examination system for animals, comprising:
a non-contact variable sensor located at a finite location, said non-contact variable sensor configured for automatically detecting medical data related to at least one health factor, about each animal in a plurality of animals, in substantially real time for each animal as each of said plurality of animals arrives at said finite location;
a processor programmed to analyze said at least one health factor medical data and determine whether each animal of said plurality of animals is healthy or unhealthy as defined by a predetermined software function for said health factor; and
a pass-fail interface in electronic communication with said processor, comprising at least one pass indicator automatically activated when a health factor of an animal as sensed by said sensor and analyzed according to said software function indicates a healthy animal, and at least one fail indicator automatically activated when said health factor of another animal as sensed by said sensor and analyzed according to said software function indicates said another animal is experiencing an unhealthy state according to said software function, as said health factor is sensed when each of said plurality of animals moves into said finite location.

15. The system of claim 14, wherein said pass indicator is a green light and said fail indicator is a red light.

16. The system of claim 14, wherein said health factor medical data being sensed is an animal's body temperature.

17. The system of claim 16, wherein said software function is a comparison of said animal's sensed body temperature to a predefined normal body temperature for said animal, and when said sensed body temperature is found to be above said normal body temperature for said animal, said pass-fail interface activates said fail indicator.

18. The system of claim 1, wherein said processor is configured to automatically communicate a message to a user when said medically useful information includes information that said animal is experiencing one or more problems.

19. The system of claim 18, wherein said one or more problems includes stomach gurgling.

20. The system of claim 18, wherein said one or more problems includes respiratory distress.
